(19) United States
(12) Patent Application Publication
(10) Pub. No.: US 2025/0331736 A1
(43) Pub. Date: Oct. 30, 2025

(54) INFERRING LUNG FUNCTION DIRECTLY FROM HEART SIGNALS

(71) Applicant: The Trustees of Dartmouth College, Hanover, NH (US)

(72) Inventors: Kofi Odame, Lebanon, NH (US); Maria Nyamukuru, West Lebanon, NH (US)

(21) Appl. No.: 19/191,895

(22) Filed: Apr. 28, 2025

Related U.S. Application Data
(60) Provisional application No. 63/639,394, filed on Apr. 26, 2024.

(51) Int. Cl. A61B 5/091; A61B 5/349

(52) U.S. Cl. CPC A61B 5/091; A61B 5/349

(57) ABSTRACT

Systems and methods for lung function parameters are disclosed herein. An electrocardiographic (ECG) device acquires ECG signals from a patient during a breathing cycle. A neural network trained to perform ECG-derived respiratory (EDR) analysis extracts respiratory information from the acquired ECG signals. A processor accesses the neural network. Respiratory features are extracted from the ECG signals. A forced expiratory volume (FEV1) of the patient during the breathing cycle is determined using the respiratory features. A report of lung function is generated using the FEV1.

### PRIORITY CLAIM

The present document claims priority to U.S. Provisional Patent Application, 63/639,394, filed Apr. 26, 2024. The entire contents of the aforenamed provisional patent application are incorporated herein by reference.

### BACKGROUND

Airway obstruction in obstructive lung diseases, including chronic obstructive pulmonary disease (COPD) and asthma, is not constant. For example, in COPD, obstruction in chronic bronchitis and emphysema may vary with time, and asthma often involves “attacks” where obstruction may be far more severe than the patient's normal condition. Monitoring lung airway obstruction—a condition where inflammation and excess mucus production in the lungs' airways impedes airflow—is critical for early detection of symptom flare-ups and possible infections in obstructive lung disease, in COPD these flare-ups and infections are known as exacerbations. If we detect and treat these attacks or COPD exacerbations early, it will increase patients' quality of life, reduce mortality, and potentially eliminate billions of dollars in healthcare costs because exacerbations and asthma attacks can lead to hospitalizations that can sometimes be avoided with prompt outpatient treatment. It is also desirable to monitor patients to determine whether medication dosages and types are appropriate or need modification.

It is important to monitor asthma-related obstruction because patient reports and symptom logs are often inaccurate, and because symptoms often vary with activity, environmental conditions including inhaled allergens, food allergens, and viral infections. Asthma can not only lead to infections and result in expensive emergency room visits, but severe asthma attacks can themselves be fatal. Asthma is often controlled with a combination of short-acting medications such as beta agonists, and longer-acting “controller” medications such as inhaled or oral steroids that frequently have undesirable side effects. With good patient monitoring asthma medications can be adjusted to both minimize undesirable side effects of medications and keep attack frequency and pulmonary obstruction to tolerable levels.

Ideally, early detection of COPD exacerbation and treatment by monitoring, or monitoring of asthma, requires measurements be made outside medical facilities, such as at home.

The state-of-the-art for measuring lung function at home is hand-held spirometry, a technique hindered by low patient adherence and often by poor patient technique. In response, some authors propose to monitor lung health with mobile and wearable technology. Approaches taken include physical activity monitoring, and accelerometer-based assessment of cardiorespiratory function. These approaches can distinguish between healthy controls and patients with lung disease, but they provide no information on a patient's severity level of lung airway obstruction-a continually changing measure that is critical for assessing disease progression and exacerbations.

A few researchers have explored ways to infer severity of airway obstruction from non-spirometric data, such as CT scans, that cannot be measured with a wearable device. As they are not wearable, they are unsuitable for continuous patient monitoring.

There is a growing infrastructure of smartwatches, smartphones and wearable devices that can measure heart activity via electrocardiogram photoplethysmography (PPG). It is also possible to measure heart activity with bioimpedance sensors. Some PPG devices can also measure breathing rate. While breathing rate can provide a coarse picture of lung health, it is inadequate for inferring more detailed information, like the extent and severity of lung airway obstruction. Respiration affects the rhythm and electrical activity of the heart; these changes can be sensed with ECG or PPG or a combination of ECG and PPG and used to extract breathing rate from the ECG and PPG signals.

For purposes of this document, “heart signals” include heart activity signals sensed with ECG, PPG, electrode plates, or bioimpedance sensors.

A person experiencing lung airway obstruction will take longer than usual to exhale, as illustrated in FIG. 1. This is reflected in an abnormally short fractional inspiratory time (FIT). FIT is defined as the duration of the inspiratory phase as a fraction of the total respiratory period. FIT drops from a normal range of 0:45-0:5 to as low as 0:2 in the presence of severe airway obstruction.

Traditionally, lung function is measured by spirometry in laboratories. Spirometry reports measures including Forced Vital Capacity (FVC), Forced Expiratory Volume (FEV), Forced Expiratory Volume in one second (FEV1), and ratios like FEV1/FEV that are of diagnostic significance to pulmonologists and other physicians.

### SUMMARY

In some aspects, the present disclosure can provide a system for estimating lung function parameters. The system can include an electrocardiogram (ECG) device, a neural network, and a processor. The ECG device can acquire ECG signals from a patient during a breathing cycle. The neural network can be trained to perform ECG-derived respiratory (EDR) analysis to extract respiratory information from the acquired ECG signals. The processor can access the neural network and carry out steps, including extracting respiratory features from the ECG signals. A forced expiratory volume (FEV1) of the patient during the breathing cycle can be determined using the respiratory features. A report of lung function can be generated using the FEV1.

In further aspects, the present disclosure can provide a lung function monitoring device. The device can include one or more electrodes, an electronic neural network, and a processor. The one or more electrodes can derive electrocardiogram (ECG) data from a patient during a breathing cycle. The processor can receive the ECG data from the one or more electrodes and access the neural network. A skewness of the ECG signal can be extracted. A force expiratory volume in one second (FEV1) or a forced vital capacity (FVC) can be determined using the skewness. A lung function can be determined based on the FEV1 or the FVC. A report of the lung function can be generated.

In further aspects, the present disclosure can provide a method of determining a classification of lung function based upon heart signals acquired from a subject. Heart signals can be obtained from one or more fingers of the subject. A plurality of parameters can be extracted from the heart signals. The plurality of parameters can correspond to a skewness of the heart signals. A force expiratory volume in one second (FEV1) or a force vital capacity (FEV) can be determined using the heart signals or the plurality of parameters. A report on symptoms of a lung condition of the subject can be generated using the FEV1 or the FVC.

### BRIEF DESCRIPTION OF THE DRAWINGS

FIG. 1 is an illustration of inspiratory and expiratory phases in a typical breath cycle.

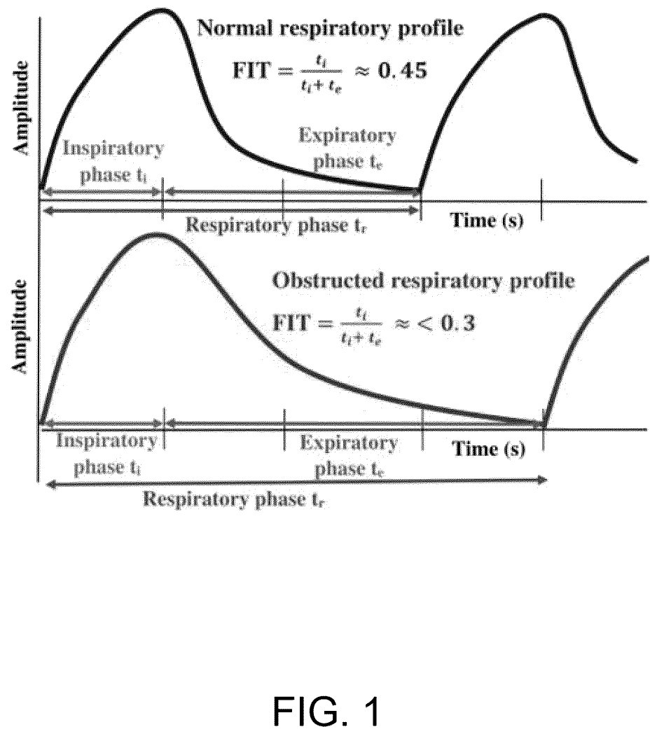

FIG. 2A is a schematic block diagram of an example system for performing patient monitoring, according to some embodiments.

FIG. 2B is a high-level data flow diagram of an example embodiment of a system for performing patient monitoring that extracts a fractional inspiratory time (FIT) and respiration rate (RR) from electrocardiogram (ECG) data, according to embodiments.

FIG. 3 is a pair of graphs illustrating parameter weights associated with a neural network, according to some embodiments.

FIG. 4 is a detailed block diagram of an embodiment of a digital signal processing (DSP) system used in a lung function monitor device with a neural network, such as a gated recurrent unit (GRU) neural network, to extract lung function parameters from electrocardiographic signals, and a classifier, such as a KNN classifier, SVM classifier, polynomial regression classifier, or a neural network classifier, to determine a level of airway obstruction, according to embodiments.

FIG. 5 is an example implementation of a mobile ECG device, according to some embodiments.

FIG. 6 is an example schematic design for a mobile ECG device used to measure ECG signals from a subject's fingers, according to some embodiments.

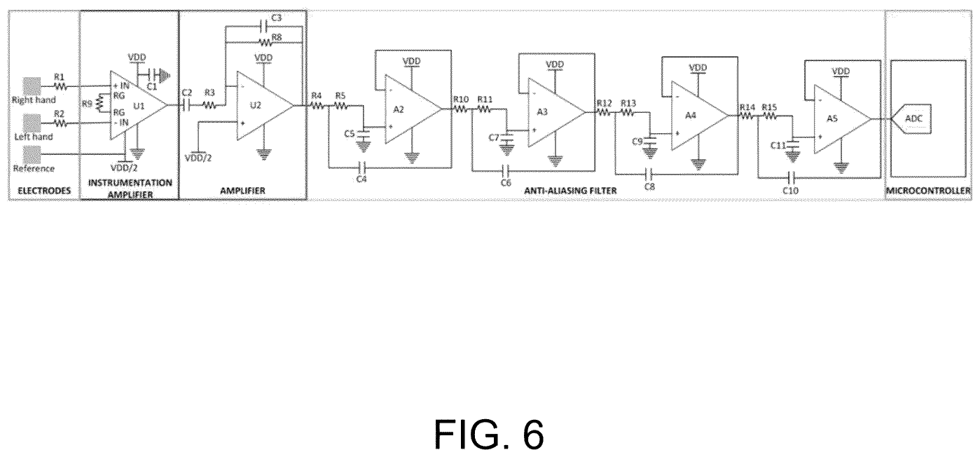

### DETAILED DESCRIPTION OF THE EMBODIMENTS

Remote and continuous monitoring of lung function is useful for the early detection of and monitoring of respiratory diseases. Lung function monitoring is mostly estimated from respiratory signals. Respiration affects heart signals, and as such, we derive measures of lung function from heart signals measurable in a wearable device. The heart signals are obtained from a subject or patient wearing the device. The device monitors lung function by estimating important pulmonary function parameters from the heart signals. The pulmonary factors assessed include some or all of fractional inspiratory time (FIT), inspiratory/expiratory ratio (I/E ratio or IER), forced vital capacity (FVC), forced expiratory volume (FEV), forced expiratory volume in one second (FEV1), and tidal volume (TV). These parameters are used to monitor lung function and infer a degree of airway obstruction. The heart signals used to estimate these parameters include Electrocardiogram (ECG) signals, may include photoplethysmogram (PPG) (sometimes known as pulse oximetry) signals, and, in some embodiments, may include bioimpedance images of heart-related movements. Specifically, features of an ECG or ECG-derived respiratory signals (EDR) signal are extracted to predict respiratory parameters.

Alterations to the ECG between expiratory and inspiratory phases of the respiratory cycle in turn produce subtle changes to the ECG signal. For instance, the amplitude envelope of the ECG's “R peaks” is modulated during the breathing cycle due to changes in the heart's position as the diaphragm expands and contracts. When airway obstruction increases the exhalation time, this is reflected by a longer positive half cycle in the ECG's amplitude modulation envelope. Also, the heart rate increases during inhalation and decreases during exhalation due to pressure changes in the thoracic cavity. So, longer-than-usual exhalation times caused by airway obstruction produce corresponding longer periods of lowered heart rate that are observable in the ECG or PPG signal. Similarly, oxygen saturation levels observed by PPG may vary through the respiratory cycle.

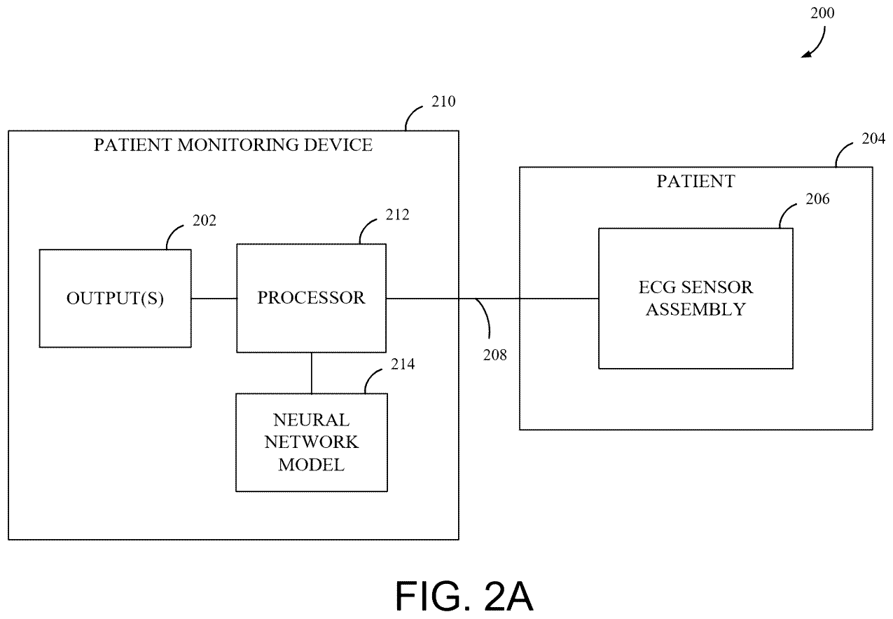

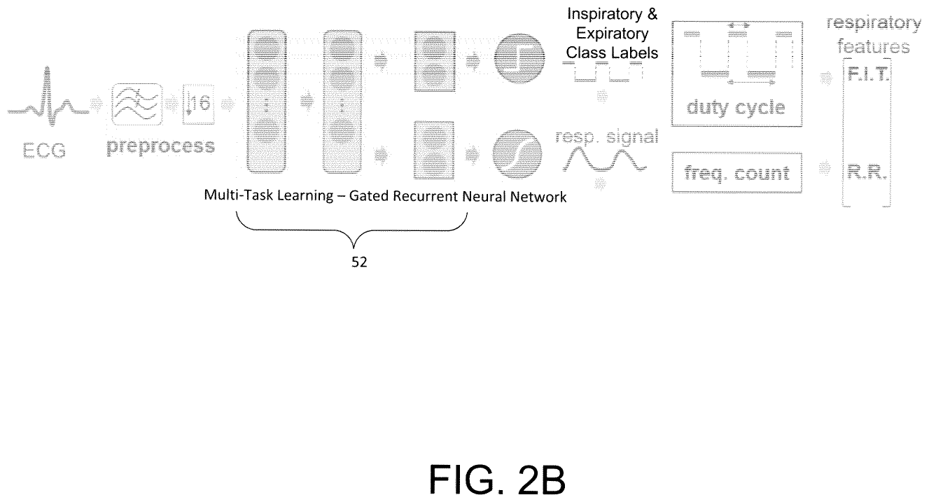

Referring to FIG. 2A, an example monitoring system 200 that can be used to perform patient monitoring and diagnosis is illustrated. A patient monitoring device 210 can be a computer, a computing chip, or any suitable computing device with a processor 212 and, optionally, one or more outputs 202. As will be described, the patient monitoring device 210 can predict respiratory parameters and diagnoses directly from heart signals and other non-respiratory electrical measurements.

In the monitoring system 200, a medical patient 204 is monitored using one or more inputs, such as an ECG sensor assembly 206, each of which transmits a signal over a communications link 208, which may be wired or wireless, to the patient monitoring device 210. In some aspects, an input (not shown) may be configured to receive an indication from a user. The ECG sensor assembly 206 can include physiological sensing elements such as, for example, ECG electrode sensors, finger pads, a chest belt, wearable electromagnetic sensors, or the like. The ECG sensor assembly 206 can generate predicted heart signals and other cardiovascular parameters of the patient 204. The signals are then processed by the processor 212. In some configurations, the processor 212 can be any suitable hardware processor or combination of processors, such as a central processing unit (CPU), a graphics processing unit (GPU), and so on, or a combination thereof. The processor 212 then communicates the processed signal to the output 202, such as a display. In some configurations, the output 202 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on, and/or non-visual outputs, such a auditory systems that communicate reports or alerts or the like. In some configurations, the output 202 can be incorporated in the patient monitoring device 210. In other configurations, the output 202 can be separate from the patient monitoring device 210. The monitoring system 200 can be a portable monitoring system in one configuration. In another instance, the monitoring system 200 can be a single assembly, without a display, and is adapted to provide respiratory diagnosis information to a remote display.

In some configurations of the system shown in FIG. 2A, the hardware used to receive and process signals from the sensors can be housed within the same housing. In other configurations, some of the hardware used to receive and process signals can be housed within a separate housing. In addition, the patient monitoring device 210 can include hardware, software, or both hardware and software, whether in one housing or multiple housings, used to receive and process the signals transmitted by the ECG sensor assembly 206.

As further illustrated in FIG. 2A, the processor 212 may further access a neural network model 214. In some examples, the neural network model may be stored on any suitable storage device or devices, such as a memory embedded within the patient monitoring device 210. The neural network model 214 may be a feedforward neural network (FNN), a convolutional neural network (CNN), a recurrent neural network (RNN), a generative adversarial network (GAN), or any other type of neural network. In some examples, the neural network model 214 may contain one or more models that may be trained using different data and may be used to identify different parameters based on information received from the ECG sensor assembly 206.

In one example, the neural network model 214 within the patient monitoring device 210 may be adapted to extract inspiratory and expiratory class labels from based on the signal received from the ECG sensor assembly 206, as illustrated in FIG. 2B. As illustrated in FIG. 2B, the neural network model 214 may be a gated recurrent neural network that extracts the class labels. The neural network model 214 can provide a phase class output indicating inspiratory phase versus expiratory phase that is used to calculate the fractional inspiratory time (FIT) and respiratory rate. The neural network 214 can also output an estimated tidal volume (TV). The FIT is calculated for every complete breathing cycle, as is a respiratory rate (RR). A complete breathing cycle includes an entire inspiration phase and expiration phase. The FIT provides a direct measurement of airway obstruction and is combined in a classifier with the respiratory rate and tidal volume to estimate the lung function parameters; forced expiratory volume (FEV), forced expiratory volume in one second (FEV1), forced vital capacity (FVC), and tidal volume (TV); and to estimate a severity of a subject's obstructive lung disease, including severity of COPD, asthmatic obstruction, or other lung disease. In alternative embodiments, the inspiratory phase and expiratory phase signal(s) are used to generate an inspiratory/expiratory ratio (I/E ratio or IER) that is used in place of FIT as a measurement of airway obstruction in estimating other measures of lung function and estimating a severity of a subject's COPD. In embodiments, the classifier is a K-Nearest-Neighbors (KNN) classifier. In alternative embodiments, the classifier is another trainable classifier such as a support vector machines (SVM) classifier, a second neural network, or a polynomial regression-based classifier.

Referring again to FIG. 2A, in another example, the neural network model 214 of the patient monitoring device 210 may be adapted to predict FEV1 and FVC, or other respiratory or physiological parameters, directly from the signals received from the ECG sensor assembly 206. That is, as will be explained, the present disclosure recognizes that conditions, such as airflow obstruction, as measured by FEV1, reflect significant changes in respiratory patterns during tidal breathing. These changes to the tidal breathing signals can be discerned via FEV1 and be used to determine, quantitatively, FEV1. Further, the present disclosure recognizes that respiratory signal influences ECG signals in multiple ways and, therefore, features of FEV1 embedded within the tidal respiratory signal can, in turn, be reflected in the ECG data. With these realizations, the present disclosure provides systems and methods that determine FEV1 and FVC from non-respiratory signals measured during tidal breathing.

Returning to FIG. 2A, the neural network model 214 may be trained to identify feature sets for predicting FEV1 and FVC using electrical signals acquired from the patient 204, such as ECG signals. In one non-limiting example, the systems and methods provided herein can use the neural network model 214 to analyze electrical or other non-respiratory physiological signals. For example, the neural network model 214 may be configured to analyze an ECG or other type of heart signal to determine FEV1 and/or FVC, or other information. For example, the neural network model 214 may perform FEV1 prediction via a feature combination that uses EDR signals. As such, the FEV1 prediction may be based on a weighted combination of feature analysis. Some non-limiting examples of features may include inspiratory-expiratory ratio (IER), expiratory reserve amplitude (ERA), inspiratory reserve amplitude (IRA), tidal amplitude (TA), respiratory rate (RR), expiratory amplitude in one second (EA1), skewness(S), detrended fluctuation analysis (DFA), and sample entropy (SE).

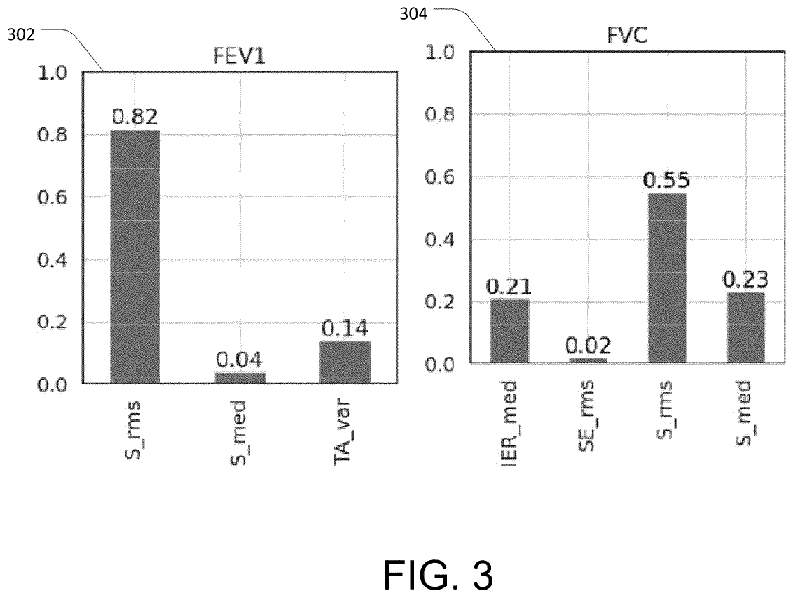

As one non-limiting example, this may include the root mean square (rms) of the skewness (Sᵣₘₛ), the median of the skewness (S_{med}), and/or the variance of the tidal amplitude (TAᵥₐᵣ) as determined from the ECG or EDR signal. In one non-limiting example, Sᵣₘₛ may contribute a weight of 0.82 to the FEV1 regression model. In this non-limiting example, TAᵥₐᵣ may have a weight of 0.14, and S_{med} may contribute with a weight of 0.04. For FVC estimation, a similar or different feature combination and/or weighting may be used. In one non-limiting example, features of the ECG or EDR signals for FEV determination may include median of the inspiratory-expiratory ratio (IER_{med}), skewness (S_{med}), rms of the sample entropy (SEᵣₘₛ) and/or rms of skewness (Sᵣₘₛ). In one non-limiting example, Sims can have a weight of 0.55, S_{med} can have a weight of 0.23, IER_{med} can have a weight of 0.21, and SE_{med} can have a weight of 0.02. The relative weights of each feature within this non-limiting example for determining the FEV1 and FVC regressors are visualized in FIG. 3. The left subplot 302 displays the feature weights for the FEV1 regressor, while the right subplot 304 shows the weight for the FVC regressor.

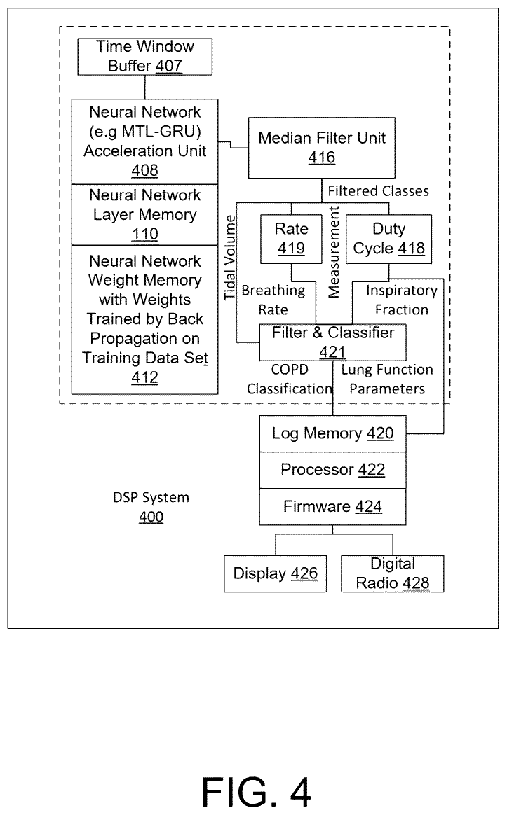

FIG. 4 is a block diagram of one non-limiting example of a digital signal processing (DSP) system for a patient monitoring device, such as the patient monitoring device 210 described above with respect to FIG. 2A. For example, the DSP system 400 may be embedded with the patient monitoring device 210. As described above, the patient monitoring device may receive electrical signals originating from the subject's heart from one or more sensors (e.g., ECG sensors assembly 206). In some examples, the signals may be preprocessed through a data acquisition unit as digitized heart signals to the digital signal processing (DSP) system 400 that performs data logging, data processing, and data export.

In one example, the DSP system 400 takes the digitized heart signals as input and may classify the inspiration and expiration phases of the underlying respiratory signals from the heart signals. In another example, the DSP system 400 may be used to predict respiration parameters directly from the digitized heart signals, without extracting inspiration and expiration phases. In embodiments, the classification of heart signals may be analyzed using a neural network 408, that, in embodiments, is a Gated Recurrent Unit (GRU) neural network that has three hidden GRU layers followed by two fully connected (FC) layers; other embodiments may have four, five, or more GRU layers. In additional embodiments, neural network 408 is a convolutional neural network or a long short-term memory (LSTM) neural network, used to extract inspiratory and expiratory phases from the heart signals. In yet other embodiments, other techniques such as heuristic algorithms may be used to extract inspiratory and expiratory phase information from the digitized heart signals.

The difference in amplitude of ECG R waves between inspiratory peaks and expiratory valleys is a function of respiratory tidal volume (TV); this and other effects permit training the neural network 408 to extract an estimated TV from the heart signals. In some configurations, the neural network 408 is a GRU neural network trained on temporally aligned heart signals, TV, and a signal representing two phases of respiratory signals, where a first phase signal represents the inspiration phase, and a second phase signal represents the expiration phase of respiration during which the temporally aligned heart signals were recorded. In embodiments, the temporally aligned heart signals and respiratory signals for training the neural network are obtained from a library of heart signals and respiratory measurements and signals obtained from a variety of patients having a range of obstructive lung symptoms from normal to severe obstruction. In a particular embodiment, in addition to training the GRU neural network on a training set of temporally aligned heart signals and respiratory signals obtained from a library of heart signals with corresponding spirometric measurements of tidal volume, FEV1, and FVC, the GRU neural network is also trained on heart signals and respiratory signals obtained from a particular subject or patient for whom the device is being configured and for whom the device is prescribed and who will be the subject or patient wearing the device. Once the network is trained, typically on a workstation computer, the weights determined are saved in a neural network weight memory 410 of the device.

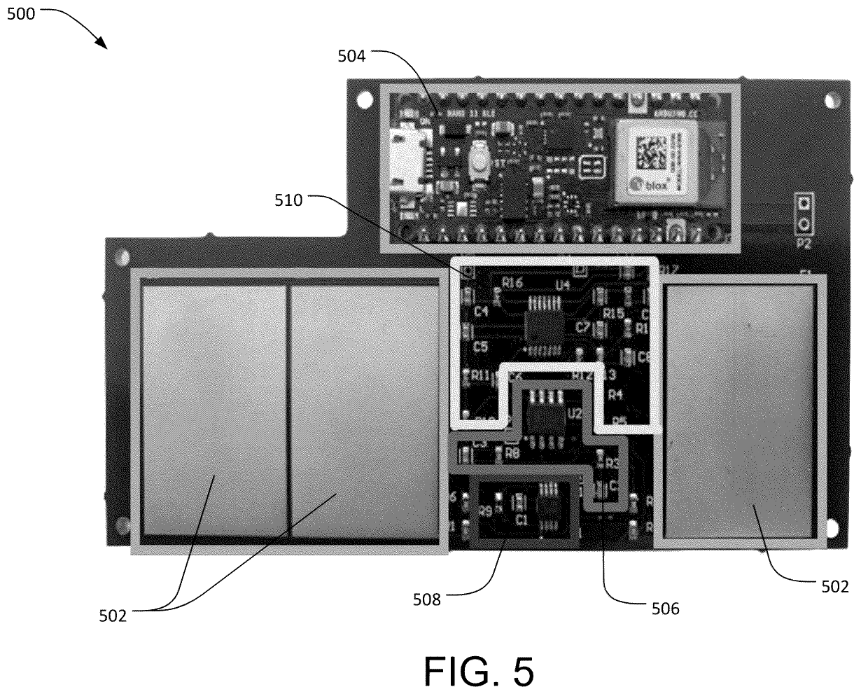

FIG. 5 illustrates an example implementation of a mobile ECG device 500. The mobile ECG device may include a set of electrodes 502, a microcontroller 504, an amplifier 506, an instrumentation amplifier 508, and an anti-aliasing filter 510. In some examples, the mobile ECG device 500 may correspond to the one or more sensors 102 and the data acquisition unit 106, as described above with respect to FIG. 3.

The electrodes 502 are sensors on which a subject's fingers are placed and may be made using exposed copper pads. In some examples, three electrodes are used: two electrodes to measure the differential ECG signal across the chest, and the third electrode as a reference electrode for common mode detection. The forefinger on the right hand can be placed on one of the electrodes, the forefinger on the left hand can be placed on the other electrode and the middle finger of the left hand can be placed on the reference electrode. The signals from these electrodes are fed into the instrumentation amplifier 508.

In some examples, the differential ECG signal being measured from the fingers can have an attenuated amplitude that ranges in the 100's of μV. This necessitates a gain of roughly 10,000 to amplify the ECG signal to Volts in order to accurately sample and analyze the signal. This small μV differential ECG signal being measured from high impedance electrodes is also buried in 60 Hz noise. Therefore, a high common mode rejection ratio and a high input impedance amplifier design is utilized. The instrumentation amplifier 508 design meets these requirements to extract the differential ECG signal from the high impedance electrodes 502, while minimizing 60 Hz noise, and providing high gain. In some examples, an additional stage of amplification is needed. For two stage amplification, most of the gain comes from the first stage in order to minimize the noise in the signal. For example, a gain of 600 can be selected for this first stage, maintaining the stability of the INA333, while providing most of the gain. As a result, the ECG signal is amplified to the mV range. This amplified ECG signal is then fed to the second amplification stage.

In some examples, the amplifier 506 takes the ECG signal as its input, and further amplifies it with a gain of 30 to achieve a total gain of roughly 18,000 which places the output ECG signal in the Volt range, and centered at the mid-rail voltage. The amplified ECG signal is then fed into an anti-aliasing filter.

In some examples, the frequency range of interest for the ECG signal is 0.05-45 Hz. For example, a conservative antialiasing filter 510 with a cutoff frequency of 48 Hz and a stop-band frequency of 150 Hz with −78.3 dB attenuation may be selected. The amplified signal is passed through this anti-aliasing filter 510, and the filtered signal is sampled by an ADC.

In some examples, the noise contribution from the instrumentation and operational amplifiers 506, 508 may be estimated using the n²ₐₘₚ equation shown below where Sᵢₙ is the noise voltage density, f_{L} is the lowest frequency required to be measured, f_{H} is the highest frequency required to be measured, and ₙₐₘₚ is the estimated noise. The estimated ADC noise is used to determine the minimum effective number of bits (ENOB) required for our ADC using the equations shown below.

$${n}_{amp}^{2} = {\int }_{{f}_{L}}^{{f}_{H}} {S}_{in}^{2} ( f ) df$$

$${n}_{tot}^{2} < {n}_{INA 333}^{2} + {n}_{AD 8607}^{2} + {n}_{AD 8609}^{2} + \frac{{n}_{ADC}^{2}}{{Gain}^{2}}$$

$${n}_{ADC}^{2} < {( 600 \cdot 30 )}^{2} \cdot ( {0.5}^{2} - 45 * ( {0.05}^{2} + {0.025}^{2} + {0.027}^{2} ) )$$

$${n}_{ADC} < 4980 μ Vrms$$

$$SNR > 20 {log}_{10} \frac{3.3 V}{s \sqrt{2} \cdot {n}_{ADC}}$$

$$ENOB > \frac{SNR}{6} = 8 bits$$

The minimum effective number of bits for ADC is estimated to be 8 bits. An ADC that has 12 bit resolution is selected. The ADC samples the ECG signal.

The microcontroller 504 processes the digitized ECG signals which are used to classify the airflow obstruction severity or classify a lung restriction. For example, the prototype illustrated in FIG. 3 was built using the Arduino Nano 33 BLE microcontroller board, which has a low-power Arm M4 microprocessor with an in-built ADC on-board. The hardware described above is implemented as shown in the schematic in FIG. 5, and results in the portable device prototype shown in FIG. 4. The mobile ECG device 500 saves the ECG signal in 90-second buffers, which are transmitted via Serial before the next 90-second buffer is transmitted. This device 500 is used to record ECG signals under tidal breathing conditions from which the lung function parameters FEV1 and FVC are estimated.

In some examples, the mobile ECG device 500 uses dry copper electrodes 502 to measure the ECG signals, as opposed to gel electrodes which have lower skin-electrode impedance and maintain good contact with the skin during movements. The use of dry electrodes 502 in the mobile ECG 500 device makes it sensitive to limb movement and requires the user to always make complete contact with the electrodes 502 during data collection. To ensure the reliability of the mobile ECG device 500 data collection, a pre-processing step involving ECG signal quality assessment is implemented. This step identifies and discards noisy ECG signals, retaining only high-quality data for further analysis. The details of the ECG signal quality check are described below.

For each window, the R-peaks of the ECG signal are identified using Python's BioSPPY package. If there are fewer than 12 R-peaks, the window is discarded. For those windows with more than 12 R-peaks, the average R-R peak distance is computed, and the average heartbeat size is determined. Profiles of each heartbeat are extracted by partitioning the ECG signal into windows equivalent to the average heartbeat size centered at each R-peak. Each heartbeat profile is then normalized by dividing by the Euclidean norm of the heartbeat. An average heartbeat template is then computed as the mean of all the heartbeat profiles. The correlation between each heartbeat profile and the average template is computed, and the average correlation across all heartbeats is found. If the average correlation is less than 0.78, the ECG window is considered noisy and is discarded. 0.78 was determined empirically. ECG windows that fail the quality check are discarded.

What makes this device novel is the estimation of FIT, followed by determination of obstructive lung symptoms severity, from ECG or other heart signals.

The extraction of FIT from heart signals makes it such that we can use existing and prevalent technology and devices that are used to monitor heart signals to also monitor lung function.

Heart signal sensors are typically noisy, which jeopardizes the FIT estimation and therefore reduces the accuracy of the lung function monitoring. This challenge can be addressed by designing robust digital filters and increasing redundancy in the heart signal sensors. These improvements are projected to result in an estimation error that is comparable to spirometry, the current gold standard.

As a technology for analyzing pulmonary signs, the invention is useful in a broad range of respiratory disease applications, such as COVID-19 and other pneumonia monitoring and treatment, lung transplant post-operative care, and respiratory therapy research, that require continuous, objective, and unobtrusive monitoring of pulmonary function changes. Since the invention is compatible with low-cost microcontrollers, it has the potential to dominate the spirometry market and respiratory clinical trials space with high-volume production.

In an exemplary embodiment of a patient monitoring device 210 (e.g., as shown in FIG. 2A), at least two, and in some embodiments only two, electrodes are positioned on a subject (not shown) in positions where they can acquire electrocardiographic (ECG) signals from the subject. The ECG signals are fed to an ECG unit where they are amplified and digitized in a data acquisition unit; in a particular embodiment these signals are digitized into digital ECG signals at 30 analog-to-digital conversions per second. These signals are fed to a data DSP unit (e.g., DSP system 400) where they are captured in a time window buffer 407. In a particular embodiment, the ECG signals are filtered by a bandpass filter having a low frequency cutoff of 0.05 hertz and a high frequency cutoff of 50 hertz before being captured in the time window buffer 407. The time window buffer 407 collects digitized ECG signals for a time window expected to be longer than most breathing cycles. In an embodiment, the time window buffer 407 may be 20 seconds to a minute long and, in a particular embodiment, 25 seconds long.

Digitized ECG signals for each time window are fed to a neural network that, in a particular embodiment, is a Multi-Task Learning-Gated Recurrent Unit (MTL-GRU) neural network 408 that is implemented in software in some embodiments and may, in some embodiments, be implemented in a hardware neural network acceleration unit. The neural network includes a layer memory 410 that holds current values for each simulated neuron of each layer of the neural network, and a weight memory 412 configured with the weights previously determined by backpropagation during training with the training set on a similar neural network running on a workstation, as previously described. The weights include weights for each synapse of the neural network. The neural network provides classified signals indicative of inspiration and expiration breathing phases to a median filtering unit 416 that provides filtered class labels to a duty cycle measurement unit 418, which provides a FIT. After the digitized heart signals of an entire time window are fed to the neural network, the neural network state memory is reset for the next time window. Median filtering unit 416 filters class labels, breathing rates, and tidal volume across multiple time windows.

In alternative embodiments, the GRU neural network is replaced with either a convolutional neural network or a long short-term memory (LSTM) neural network. In these alternative embodiments, as with the GRU neural network, each neural network has at least three layers, and in embodiments 3, 4, 5, or 6 layers, all configured with synapse weights in a weight memory; the synapse weights determined by back propagation in a similar neural network executing on a workstation while training on the training set previously described.

The sum of inspiratory time and expiratory time provides a measure of respiration period, which can be inverted in a rate measurement unit 419 to provide a respiration rate RR. Measured duty cycles provide breathing-related signals including an inspiratory fraction FIT and breathing rate, along with the tidal volume TV, to a data logging memory 420 where they are recorded with a current time tag. ECG signals from ECG unit 404 or PPG signals, may also have pulse rate and other characteristics such as arrhythmias measured and logged in data logging memory 420. In addition to logging TV, FIT and RR, FIT, TV, and RR or respiration period are input to a classifier 121 that filters measured TV, FIT and RR and compares measured TV, FIT and RR against TV, FIT and RR determined from other recent breathing cycles to drop TV, FIT and RR measurements that are anomalous, then classifies the filtered TV, FIT and RR or respiratory period as representative of mild, moderate, severe, or very severe lung obstruction, or of mild, moderate, severe, or very severe COPD. In embodiments, the classifier further derives other lung parameters, including one or more of FEV1/FEV or FEV1/FVC, FEV1, FEV, from the TV, FIT, and RR or respiration period. The lung obstruction and/or COPD classification is also logged in logging memory 420 with times of the measurements so a time progression of disease or exacerbations can be determined by a physician who downloads data from the log memory 420 through digital radio 428. As previously stated, the classifier is selected from a decision tree classifier, random forest classifiers, a KNN classifier, a SVM classifier, a neural network, a polynomial regression classifier, etc.

A processor 422 is provided with firmware 424 to control operation of the entire heart and breathing monitor device, including executing functions of the DSP or controlling operation of neural network acceleration hardware that performs the neural network functions, and to provide inspiratory fraction, breathing rate, tidal volume, pulse rate, and other arrhythmias, and the other lung function parameters selected from FEV1/FEV, FVC, and FEV1/FVC in human readable form on display 426 and in machine readable form via digital radio 428 to cell phones and computers where they may be further processed so a physician can monitor the subject's health, a server can update the subject's health records, and alert medical personnel when logged lung function parameters exceed safe limits for the subject.

In an embodiment, the GRU network has three 16-unit hidden GRU layers, one 16-unit fully connected layer, and a single unit output layer. The network processes 25-second long (7500 samples at a 300 sample-per-second sampling rate) sequences in a sampling window, the sequence sampled at 30, 300, or another rate between 30 and 300, samples per second, and returns output for each sample in the sequence. Before calculating the FIT, the duty cycle measurement unit 418 identifies the number of complete cycles (Nc) in the 25-second window. A complete cycle includes an inspiratory phase and an expiratory phase. The duty cycle measurement unit 418 computes the FIT for each complete respiration cycle FIT=Ni/Ntot where Ni is the number of samples in the inspiratory phase and Ntot is the total number of samples in the complete respiration cycle, which is the sum of samples in the inspiratory and expiratory phases of breathing.

Respiratory rate is computed for each 25-second window as Fs=60×Nc/Stot, where Fs is the sampling frequency Nc is the total number of complete cycles in each window and Stot is the total number of samples in each complete 25-second window.

Tidal volume is estimated by the neural network from differences in peak R-wave amplitude, respiratory rate, pulse rate, and pulse rate variability of ECG. We note that pulse rate, pulse rate variability, and respiratory rate can also be derived from PPG signals or bioimpedance signals and used by a neural network to estimate tidal volume.

It is desirable that the window sampled and processed to determine respiratory rate and FIT be greater in length than most breaths. In alternative embodiments, longer windows than 25 seconds are used within the range 20 to 60 seconds in width.

In one embodiment, a k-nearest neighbor (KNN) classifier is used to detect the absence or presence of lung airway obstruction, using FIT, RR and TV as the predictor variables, and applying the GOLD criteria on FEV1/FVC to obtain the true class labels. The tidal breathing parameters were also used to classify the severity of airway obstruction or lung restriction. Since this is an ordinal classification problem, in one embodiment, we used a classifier that is based on a regression model (independent variables: FIT, RR, TV; dependent variable: percent-age predicted FEV1). The regression model's estimated FEV1 score was used to classify airway obstruction severity, according to the GOLD criteria to distinguish between all four severity levels of mild, moderate, severe, and very severe obstruction.

In alternative embodiments, other forms of classifier are used, including a neural network classifier trained on extracted FIT, TV, and RR with spirometric data from the training set.

Changes may be made in the above system, methods or device without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A system for estimating lung function parameters, comprising:
an electrocardiogram (ECG) device configured to acquire ECG signals from a patient during a breathing cycle;
a neural network trained to perform ECG-derived respiratory (EDR) analysis to extract respiratory information from the acquired ECG signals;
a processor configured to access the neural network and carry out steps comprising:
extract respiratory features from the ECG signals;
using the respiratory features, determine forced expiratory volume (FEV1) of the patient during the breathing cycle; and
generate a report of lung function using FEV1.

2. The system of claim 1, wherein the processor is further configured to determine forced vital capacity (FVC) using the respiratory features.

3. The system of claim 1, wherein the processor is further configured to determine an airflow obstruction severity based on FEV1.

4. The system of claim 1, wherein the respiratory information extracted from the ECG signals includes at least one of inspiratory-expiratory ratio, expiratory reserve amplitude, inspiratory reserve amplitude, tidal amplitude, respiratory rate, expiratory amplitude in one second, skewness, detrended fluctuation analysis, and sample entropy.

5. The system of claim 1, wherein the processor is further configured to utilize a decision tree regressor trained to estimate FEV1 based on respiratory features extracted by the neural network.

6. The system of claim 1, wherein the neural network includes a convolutional neural network and a gated recurrent unit network.

7. The system of claim 1, wherein the ECG device is configured to acquire the ECG signals from fingers of the patient.

8. A lung function monitoring device, comprising:
one or more electrodes configured to derive electrocardiogram (ECG) data from a patient during a breathing cycle;
an electronic neural network;
a processor configured to receive the ECG data from the one or more electrodes and access the electronic neural network to:
extract a skewness of the ECG signal;
determine at least one of a forced expiratory volume in one second (FEV1) or a forced vital capacity (FVC) using the skewness;
determine a lung function based on the FEV1 or the FVC; and
generate a report of the lung function.

9. The device of claim 8, wherein the lung function includes at least one of airway obstruction, airway restriction, or airway inflammation.

10. The device of claim 8, wherein the report includes an indication of a severity of a lung condition.

11. The device of claim 8, wherein the lung condition includes chronic obstructive pulmonary disease (COPD).

12. The device of claim 8, wherein the processor is further configured to determine at least one of a root mean square of the skewness, a median of the skewness, and a variance of a tidal amplitude.

13. The device of claim 12, wherein the electronic neural network includes at least one of a gated recurrent unit (GRU) neural network, a convolutional neural network, and a long short-term memory (LSTM) neural network and, wherein the electronic neural network includes one or more layers of neurons and a weight memory, and wherein the weight memory contains weights determined by training a second neural network corresponding to the electronic neural network on a training data set.

14. The device of claim 13, wherein the training data set comprises temporally aligned heart signals and respiratory signals obtained from a library of heart signals and respiratory signals obtained from a variety of patients having a range of symptoms from normal breathing through symptoms including at least one lung disease characterized by obstruction.

15. The device of claim 13, wherein the training data set further comprises heart signals and respiratory signals obtained from a particular patient for whom the lung function monitoring device is being configured.

16. The device of claim 8, wherein the one or more electrodes are configured to receive a subject's fingers.

17. The device of claim 8, wherein the processor is further configured to estimate a spirometric respiratory parameter that at least includes determining a FEV1/FVC ratio.

18. A method of determining a classification of lung function based upon heart signals acquired from a subject, the method comprising:
obtaining heart signals from one or more fingers of the subject;
extracting a plurality of parameters from the heart signals, the plurality of parameters corresponding to a skewness of the heart signals;
determining at least one of a force expiratory volume in one second (FEV1) or a force vital capacity (FVC) using the heart signals or the plurality of parameters; and
generating a report on symptoms of a lung condition of the subject using the FEV1 or the FVC.

19. The method of claim 18, wherein the heart signals comprise signals provided by a sensor including at least one of an electrocardiographic (ECG) sensor, a photoplethysmographic (PPG) sensor, and a bioimpedance sensor.

20. The method of claim 18, wherein the report provides a spirometric report of the subject.
